(19) United States
(12) Patent Application Publication
(10) Pub. No.: US 2011/0152722 A1
(43) Pub. Date: Jun. 23, 2011

(54) UTERINE RUPTURE WARNING METHOD

(76) Inventor: Douglass Blaine YACKEL, Surrey (CA)

(21) Appl. No.: 12/969,052

(22) Filed: Dec. 15, 2010

Related U.S. Application Data
(60) Provisional application No. 61/282,128, filed on Dec. 22, 2009.

(51) Int. Cl. A61B 5/103

(52) U.S. Cl. 600/587

(57) ABSTRACT

An abnormal uterine wall perforation is inferred by the failure of the distal balloon of a conventional ablation apparatus, to reach a prescribed, “normal” or “expected” pressure within a prescribed time.

### CROSS-REFERENCE TO RELATED APPLICATION

This application claims priority from U.S. provisional application No. 61/282,128, filed by applicant on Dec. 22, 2009, which is incorporated herein in its entirety by reference.

### FIELD OF THE INVENTION

The invention relates to an improvement in methods and apparatus for effecting hyperthermia in a body cavity or duct.

### BACKGROUND OF THE INVENTION

The invention relates to apparatus and methods using a distal balloon or similar flexible bladder which is inserted into the uterus and filled with a heated liquid at a known pressure and for a known time to cauterize (“ablate”) the endometrium of the uterus. This method of treatment is known as “thermal balloon ablation”.

Medical treatments involving ablation of the endometrium of the uterus are well known in the prior art. The endometrium is the portion of the uterine lining to which an embryo normally attaches and is responsible for the menstrual cycles. Such ablation treatments typically involve either the direct or indirect application of heat or cold to the endometrial tissue. Commonly, ablation apparatus and techniques have been used to treat menorrhagia (a condition of excessive menstrual bleeding) by cauterizing, or inducing necrosis of the endometrial lining. This cauterization prevents further proliferation of the endometrium and may result in permanent relief of menorrhagia symptoms.

Apparatuses and methods for thermal balloon ablation are well known in the prior art. For applications to treat the endometrium of the uterus, thermal balloon ablation apparatuses typically comprise a distal, distensible balloon which is inserted into the uterus through the external opening of the cervix. The distal balloon is then inflated with a suitable liquid to expand the distal balloon such that it is in contact with substantially the entire uterine cavity. This liquid is heated and maintained at a controlled temperature for a predetermined period of time. After this period of time, the liquid is withdrawn and the distal balloon is removed from the uterus. The heat energy which is transferred from the liquid filled distal balloon to the surrounding tissues of the uterus causes the desired cauterization of the endometrium. There are many examples of such apparatus in the prior art, for examples: Stevens, U.S. Pat. No. 5,800,493; and Wallsten, U.S. Pat. Nos. 5,693,080 and 5,571,153.

One problem not well addressed by the prior art relates to an accidental perforation of the uterine wall by any prior instrumentation (for example, uterine sound or dilator) that is not known by the operator. In such case, the insertion of the thermal distal balloon ablation apparatus into the uterus may result in the balloon being pushed through the perforated uterine wall without the operator's knowledge. The subsequent performance of the ablation procedure may cause the heated distal balloon to severely damage the surrounding tissue and organs, all unknown by the operator.

### SUMMARY OF THE INVENTION

A method is disclosed for detecting a perforation in the uterine wall of a patient, comprising the steps of: filling a balloon with a liquid of a predetermined temperature suitable for ablation treatment of a patient; inserting said balloon into the uterus of the patient and inflating said balloon into the uterus and inflating it to contact substantially the entire uterine cavity; determining the pressure of said inserted and inflated balloon; detecting if the pressure in said balloon has reached a prescribed pressure within a prescribed time that signifies normal pressure conditions; taking remedial measures if said prescribed pressure has not been reached within said prescribed time.

### BRIEF DESCRIPTION OF THE DRAWINGS

A better understanding of the present invention can be obtained when the following detailed description of the preferred embodiment is considered in conjunction with the following drawings, in which:

FIG. 1 is a perspective view of an idealized, conventional ablation apparatus; and

FIG. 2 is a schematic block diagram of the electronic controls of the apparatus of FIG. 1.

### DETAILED DESCRIPTION OF THE PREFERRED EMBODIMENT

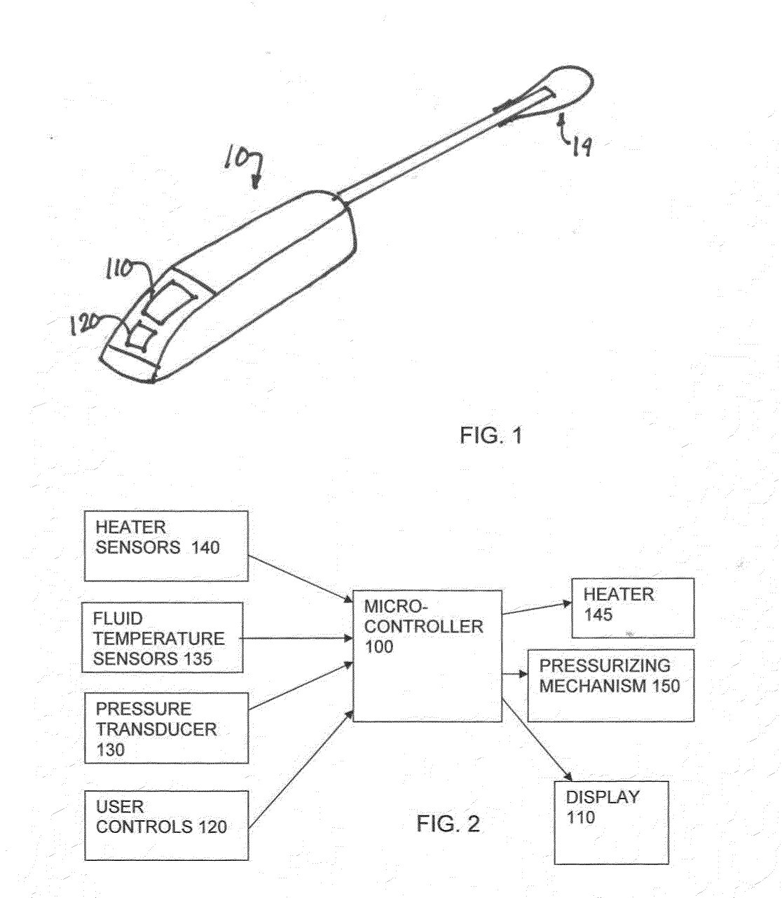

The applicant herein, has himself contributed to the prior art; in particular U.S. Pat. No. 7,419,500. The thermal balloon ablation apparatus and method described therein teaches an effective method of thermal balloon ablation. U.S. Pat. No. 7,419,500 is incorporated herein by reference as an example of conventional apparatus. As seen in U.S. Pat. No. 7,419,500 and in conventional ablation apparatus in general, and in FIG. 1 hereof in particular, a conventional ablation apparatus 10 has display 110 and controls 120, at one end for the operator, and thermal balloon 14 at the distal end, with management functionality and related electro/mechanical components (some of which is not shown). Display 110 has conventional audio-visual components to inform and warn the operator.

More particularly, apparatus 10 has a distal flexible bladder or balloon; a proximal flexible bladder or balloon; a single-lumen catheter joining said distal and proximal flexible balloons in a liquid-tight system, a liquid sealed inside the system to flow between the two bladders; where the liquid is established to permit the distal balloon to substantially deflate when the liquid is moved out of the distal end; and wherein the catheter has two opposing ends, one end opening into the proximal balloon and the other end opening into the distal balloon, the catheter extending continuously and normally open between its ends to define an unchangeable volume for the liquid. Many of the preceding features are not shown for ease of illustration and because they are conventional and are described in more detail elsewhere (of which U.S. Pat. No. 7,419,500 is representative).

As seen in U.S. Pat. No. 7,419,500 and in conventional apparatus in general, and in FIG. 2 hereof in particular, one typical embodiment of the management function and related, has microcontroller 100 (with associated timer and electrical power supply) with operator interfaces including display 110 and controls 120 (including a power switch and an inflate/deflate switch). Microcontroller 100 accepts as input, signals from heater temperature sensors 140, liquid temperature sensors 135 and pressure transducer 130. Microcontroller 100 has outputs which manages the operation of heater 145 and pneumatic pressurizing mechanism 150. With controls 120, the operator activates (or deactivates) microcontroller 100 and all other electrical components and thereby initiates or terminates operation of the ablation apparatus and the ablation treatment. FIGS. 1 and 2, and the preceding explanations are simplified and idealized descriptions of conventional ablating apparatus and system, typified by the disclosure of U.S. Pat. No. 7,419,500. A conventional commercial example is “Thermablate Endometrial Ablation System” marketed by Woman Teoranta.

Apparatus 10 and its physical, mechanical, electrical and management control components and attributes, are conventional. The present invention teaches a new and advantageous way of using conventional apparatus 10.

The conventional ablation apparatus typically is operated according to the following conventional process. The liquid in the balloon is heated to the prescribed treatment temperature; balloon leakage and related tests are conducted, and if passed, the distal balloon 14 is inserted into the uterus until the distal end of the catheter touches the fundus, at which point, the ablation treatment is considered to be initiated.

The invention proposes to detect of a possible perforation of the uterine wall by detecting an abnormal pressure condition of the ablation apparatus distal balloon 14 and thereby inferring a perforation. Additionally, upon such detection, warning and remedial measures are taken immediately.

For example, in one embodiment (described in aforementioned example of U.S. Pat. No. 7,419,500), after a short period of time, the liquid in the distal balloon 14 reaches a steady state pressure of 180 mmHg relative to ambient pressure. At this pressure, the uterus will be fully distended and the distal balloon 14 will be filled with heated liquid and be in contact with substantially all of the walls of the uterine cavity. This is the normal treatment pressure scenario. But in case the uterine wall has been perforated and the distal balloon 14 has been unknowingly inserted through the perforation, the pressure will not reach 180 mmHg within a specified time (because the balloon is not constrained by the uterine wall as it normally would be).

If the pressure does not reach 180 mmHg within a prescribed time after ablation treatment has been initiated, microcontroller 100 is programmed to warn the operator of a possible perforation and/or to immediately take remedial measures (e.g. automatically terminate operation of the ablation treatment and deflate the balloon). In other words, the inference of a possible perforation is made and remedial steps are taken.

For another example, to minimize cooling of the liquid in distal balloon 14, microcontroller 100 pulses the pressure within the balloon system during the treatment period to initiate flow back and forth between distal balloon 14 and proximal balloon (not shown) to continually mix the volume of heated liquid. So, after initiation of treatment, the distal balloon 14 will be cyclically inflated (and deflated); and if the pressure does not reach 180 mmHg, within two cycles, microcontroller 100 is programmed to warn the operator of a possible rupture and/or to immediately take appropriate remedial measures, as mentioned above.

In some embodiments, two cycles may be in the order of 10 seconds each, and so microcontroller 100 is programmed to warn the operator of a possible perforation and/or to take immediate remedial measures if 180 mmHg pressure is not reached within 20 seconds of the initiation of the ablation treatment. In some embodiments, only three cycles are performed (the first two cycles lasting in the order of 30 seconds each, and the third cycle lasting 60 seconds); and in such a case, if the expected pressure is not presented by 10 seconds of the first cycle, the inference of perforation and warning should be made.

The above references to 180 mmHg pressure and prescribed times or cycles, are mentioned as examples only and in a non-limiting ways. The actual values (of prescribed pressure, time and time-equivalents) beyond which (or below which, as the case may be) an abnormal pressure condition is inferred, can be easily prescribed based on conventional experiments by the average skilled person in the art, based on the equipment used, size of balloon, size of the uterine cavity and the like.

The point of the invention is to infer an abnormal uterine wall perforation by the failure of the distal balloon 14 to reach a prescribed, “normal” or “expected” pressure within a prescribed, “normal” or “expected”, time, or more generally, failure to reach normal conditions. What is prescribed are physical attributes that are consistent with a distal balloon 14 that is properly and normally inflated within the uterine cavity. Failure to present timely the prescribed attributes implies abnormal conditions that are consistent with the distal balloon 14 having perforated or penetrated the uterine wall and inflating, partially or wholly, outside the uterus.

Although the method, system and devices of the present invention has been described in connection with the preferred embodiment, it is not intended to be limited to the specific form set forth herein, but on the contrary, it is intended to cover such alternatives, modifications, and equivalents, as can be reasonably included within the spirit and scope of the invention as defined by the appended claims.

The Abstract of the Disclosure is provided to comply with 37 C.F.R. section 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate preferred embodiment.

1. A method for detecting a perforation in the uterine wall of a patient, comprising the steps of:
a) filling a balloon with a liquid of a predetermined temperature suitable for ablation treatment of a patient;
b) inserting said balloon into the uterus of the patient and inflating said balloon into the uterus and inflating it to contact substantially the entire uterine cavity;
c) determining the pressure of said inserted and inflated balloon;
d) detecting if the pressure in said balloon has reached a prescribed pressure within a prescribed time that signifies normal pressure conditions; and
e) taking remedial measures if said prescribed pressure has not been reached within said prescribed time.

2. An apparatus for detecting a perforation in the uterine wall of a patient, comprising:
a) a balloon with a liquid of a predetermined temperature suitable for ablation treatment of a patient;
b) pressurizing mechanism that inflates and deflates said balloon;
c) pressure sensor that senses pressure of inflated balloon;
d) timer that measures if said balloon has reached a prescribed pressure within a prescribed time that signifies normal pressure conditions; and
e) an micro-controller that is programmed to operate said timer and said pressure sensor.
